(19) United States
(12) Patent Application Publication
(10) Pub. No.: US 2008/0234763 A1
(43) Pub. Date: Sep. 25, 2008

(54) SURGICAL COMPRESSION BONE SCREW

(76) Inventors: Chad J. Patterson, Shady Mill Cove, TN (US); Gary W. Lowery, Eads, TN (US)

Correspondence Address: DUANE MORRIS, LLP;IP DEPARTMENT, 30 SOUTH 17TH STREET, PHILADELPHIA, PA, 19103-4196, US

(21) Appl. No.: 11/687,401

(22) Filed: Mar. 16, 2007

(51) Int. Cl. A61B 17/04; A61B 17/58

(52) U.S. Cl. 606/315; 606/301

(57) ABSTRACT

A screw implant for stabilizing a bone material having a two separated regions has improved features that generates better compression of the two regions than conventional compression screws. The screw implant has a shaft including a longitudinal axis, a proximal portion, and a distal portion, the proximal and distal portions having proximal and distal threads thereon respectively. The minor diameter of the proximal thread is substantially equal to the major diameter of the distal thread and the proximal and distal threads have substantially the same thread profiles and same thread pitch. The proximal thread is continuous without any breaks, such as self-tapping flutes, and when the screw implant is inserted by rotation into the bone material, the proximal and distal portions threadably engage the first and second regions, respectively, to provide compression therebetween.

### FIELD OF THE INVENTION

The present invention relates to a screw implant for insertion in a bone material.

### BACKGROUND

Certain treatment of bone disorders or bone trauma involves use of compression bone screws to join or stabilize two bone fragments under compression. The compression provided by such screw implants assists in the knitting of the two bone fragments. Many examples of compression bone screws are known, each with varying degrees of efficiency in generating the desired compression force. Therefore, there is a continuing need for improved compression bone screws that can generate compression force more efficiently.

### SUMMARY

A screw implant for insertion in a bone material having a first and second regions to stabilize the first and second regions according to an embodiment is disclosed. The screw implant comprises a shaft including a longitudinal axis, a proximal portion, and a distal portion, the proximal and distal portions having proximal and distal threads thereon respectively. The proximal and distal threads each has minor and major diameters and the minor diameter of the proximal thread is substantially equal to the major diameter of the distal thread. The shaft of the implant has a threadless medial portion disposed between the proximal and distal portions and separates the proximal and distal portions apart. Preferably, the proximal and distal threads have substantially the same thread profiles, and the same thread pitch. The proximal thread is continuous without any breaks, such as self-tapping flutes, and when the screw implant is inserted by rotation into the bone material, the proximal and distal portions threadably engage the first and second regions, respectively, to provide compression therebetween.

According to a preferred embodiment, the screw implant is a compression bone screw. When the bone screw is threaded into two regions of the bone, the distal thread and the proximal thread individually threadably engage each of the two regions of the bone and stabilize the bone.

The combined features of the screw implant of the invention results in improved compression performance in that the screw will generate compression more efficiently. Such screw implant can be used in several types of surgical procedures, such as, osteotomies where two separated pieces of the same bone are involved, arthrodesis connecting two or more bones together, and graft fixation where bone and other materials are anchored in place by the screws.

### BRIEF DESCRIPTION OF THE DRAWINGS

FIG. 1 a plan view of a screw implant according to an embodiment.

FIG. 2 is an end view of the screw implant of FIG. 1 from its proximal end.

FIG. 3 is a longitudinal cross-sectional view of the screw implant of FIG. 1.

FIG. 4 is a detailed view of the distal threads of the screw implant of FIG. 1.

FIG. 5 is a detailed view of the proximal thread of the screw implant of FIG. 1.

FIG. 6 is a compression vs. screw turn plot comparing the compression generated by exemplary screw implants of the invention to the compression generated by a conventional compression bone screw.

The features shown in the above referenced drawings are illustrated schematically and are not intended to be drawn to scale nor are they intended to be shown in precise positional relationship. Like reference numbers indicate like elements.

### DETAILED DESCRIPTION

In the following detailed description of the preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the present invention.

FIG. 1 is an illustration of a screw implant 100 according to an embodiment. The screw implant 100 comprises a shaft 12 having a longitudinal axis 50, a proximal portion 30 and a distal portion 20. The proximal portion 30 has proximal screw thread 34 provided thereon and the distal portion 20 has distal screw thread 24 provided thereon. Between the threaded proximal and distal portions 30, 20 is a medial portion 10 that does not have any screw threads and is configured in a cylindrical shape with smooth finish. The threadless medial portion 10 keeps the threaded proximal and distal portions 30, 20 apart by a distance. The lengths of the proximal portion, 30, the medial portion 10, and the distal portion 20 can be varied depending on the particular intended application for the screw implant 100. The screw implant 100 may also include a cannulation 40 extending longitudinally through the full length of the screw implant.

In a preferred embodiment, the distal portion has at least two screw thread starts. In the illustrated example of FIG. 1, the distal portion 20 has thread starts. Thus, the screw thread 24 comprises two threads 24a and 24b. At the distal end 22 of the distal portion 20, flutes 21 can be cut into the distal screw threads 24a, 24b to provide the distal end 22 with self-drilling configuration. The interruptions in the distal screw threads resulting from the flutes 21 provide sharp cutting edges for self-drilling.

Unlike the distal screw threads 24a, 24b, the proximal screw thread 34 is a continuous single thread. The term “continuous” refers to the fact that the proximal screw thread does not have any flutes or notches cut into it. In many conventional compression bone screws, flutes or notches are cut into both the distal and proximal threads to aid in self-tapping or self-drilling function of the screw threads. However, by providing a proximal thread that is continuous, improvement in the compression generated by the screw implant was achieved.

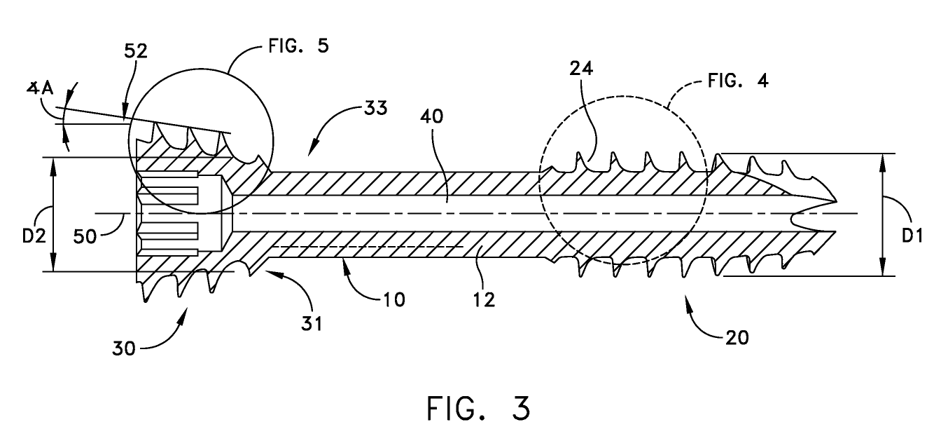

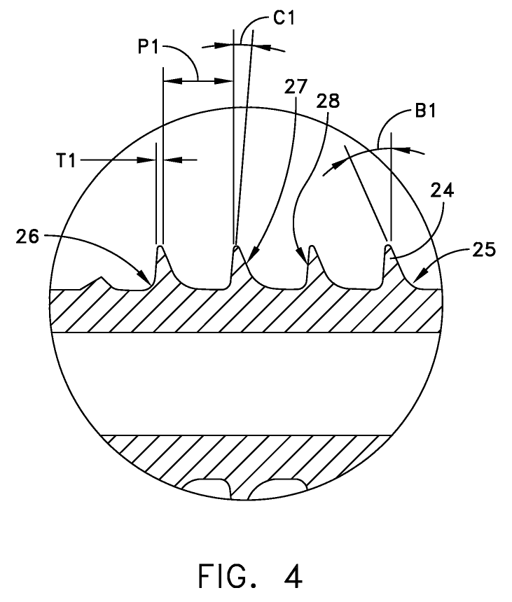

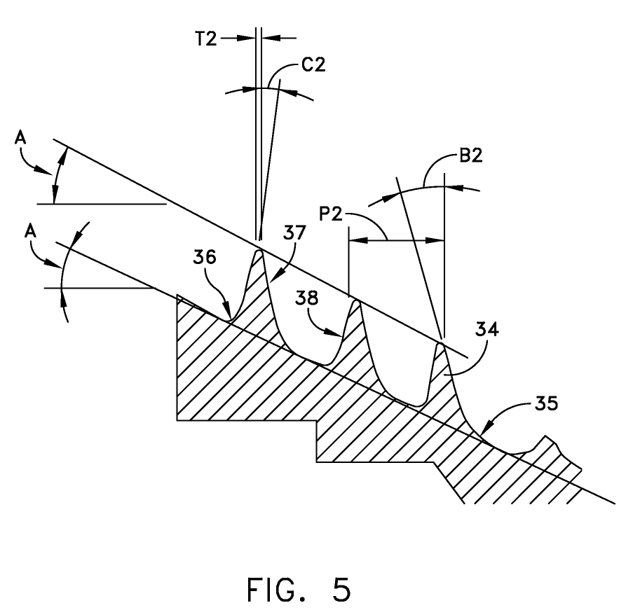

Referring to the longitudinal cross-sectional view in FIG. 3 and the detailed illustration of the distal threads 24 in FIG. 4 and the detailed illustration of the proximal thread 34 in FIG. 5, additional features of the screw implant 100 will be described in more detail. The distal threads 24 and the proximal threads of the screw implant 100 have the same thread profile. First, the pitch P1 of the distal threads 24 is same as the pitch P2 of the proximal thread 34. Secondly, thickness of the threads at the crests of the threads are same. As shown, the crest width of the distal thread T1 is substantially the same as the crest width of the proximal thread T2. Thirdly, the slope angles B1, C1 of the distal threads' leading surface 27 and trailing surface 28, respectively, are substantially the same as the corresponding angles B2, C2 of the proximal thread's leading surface 37 and trailing surface 38, respectively. The slope angles B1, C1, B2 and C2 are measured with respect to an orthogonal of the longitudinal axis 50. Furthermore, the curvatures of the trough areas between the threads are also the same between the distal threads and the proximal thread. Referring to the figures, the leading trough curvature 25 at the base of the leading surface 27 of the distal threads 24 is substantially the same as the corresponding leading trough curvature 35 at the base of the leading surface 37 of the proximal thread 34. Correspondingly, the trailing trough curvature 26 at the base of the trailing surface 28 of the distal threads 24 is substantially the same as the trailing trough curvature 36 at the base of the trailing surface 38 of the distal threads 34. The thread profile dimensions can have the following range of values: the thread pitches P1, P2=about 0.04 to 0.10 inches; the thread crest width T1, T2=about 0.002 to 0.008 inches; the slope angles B1, B2=about 20° to 30°; the slope angles C1, C2=about 2° to 8°; the leading trough curvatures 25, 35 having radius of curvature of about 0.02 to 0.04 inches; and the trailing trough curvatures 26, 36 having radius of curvature of about 0.005 to 0.015 inches. In one preferred embodiment, these thread profile dimensions are as follows: the thread pitches P1, P2=about 0.071 inches; the thread crest width T1, T2=about 0.004 inches; the slope angles B1, B2=about 25°; the slope angles C1, C2=about 5°; the leading trough curvatures 25, 35 having radius of curvature of about 0.031 inches; the trailing trough curvatures 26, 36 having radius of curvature of about 0.008 inches.

Also, as shown in FIGS. 3 and 5, the proximal thread 34 is tapered at an angle A with respect to the longitudinal axis 50 with the diameter of the proximal portion 30 increasing towards the proximal end 32, which also attributes to the enhanced compression generated by the screw implant 100. The taper angle A is measured as the slope of the tangent 52, that is tangent to the crests of the proximal thread 34, with respect to the longitudinal axis. The taper angle A can be about 3° to 12° and preferably about 5° to 8°. In a preferred embodiment, the taper angle A is about 6°.

Referring to FIG. 3, another aspect of the screw implant 100 that attributes to its enhanced compression performance is that the major diameter D1 of the distal threads 24 is substantially the same as the minor diameter D2 of the proximal thread 34. The major diameter D1 is the diameter of the distal portion 20 measured at the crests of the threads 24. The minor diameter D2 of the proximal thread 34 is defined as the diameter measured at the lowest point 33 (i.e. closest to the longitudinal axis 50) of the trough at the leading or distal end 31 of the proximal portion 30.

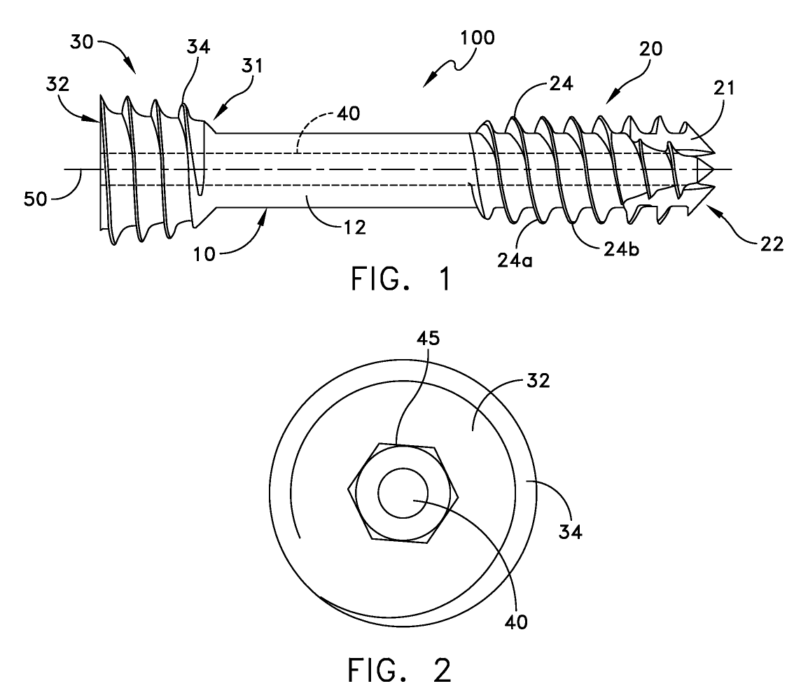

As shown in FIGS. 1 and 2, the proximal end 32 of the screw implant 100 is configured to be a flat surface, oriented orthogonal to the longitudinal axis 50. The flat surface of the proximal end 32 allows the screw implant to provide a non-protruding contour with the bone surface when fully inserted into a bone. Centrally located at the proximal end 32 is a drive tool receiving structure 45 for receiving a drive tool. The drive tool receiving structure 45 can be, for example, a slot or an appropriately shaped recess or a socket.

The various features of the screw implant 100 described herein collectively contribute to the enhanced compression performance of the screw implant 100 during insertion into a bone material having first and second regions where the proximal and distal portions of the screw implant 100 threadably engage with the first and second regions, respectively. Such bone screws generate much higher compression force than standard compression screws. The table below shows the measured compression test data on the bone screw disclosed herein compared to the compression data measured on conventional lag screw type bone screw.

COMPRESSION TESTING
(lbs.)

| | No. of screw turns | | | | | | | |
|---|---|---|---|---|---|---|---|---|
| Samples | Seated | 0.5 | 1.0 | 1.5 | 2.0 | 2.5 | 3.0 | Max |
| Reference | 2.4 | 16.1 | 24.4 | 31.2 | 33.4 | 0.0 | | 33.4 |
| A | 3.5 | 29.5 | 49.2 | 55.1 | 0.0 | | | 55.1 |
| B | 3.5 | 29.4 | 60.0 | 80.1 | 92.5 | 112.3 | 0.0 | 112.3 |

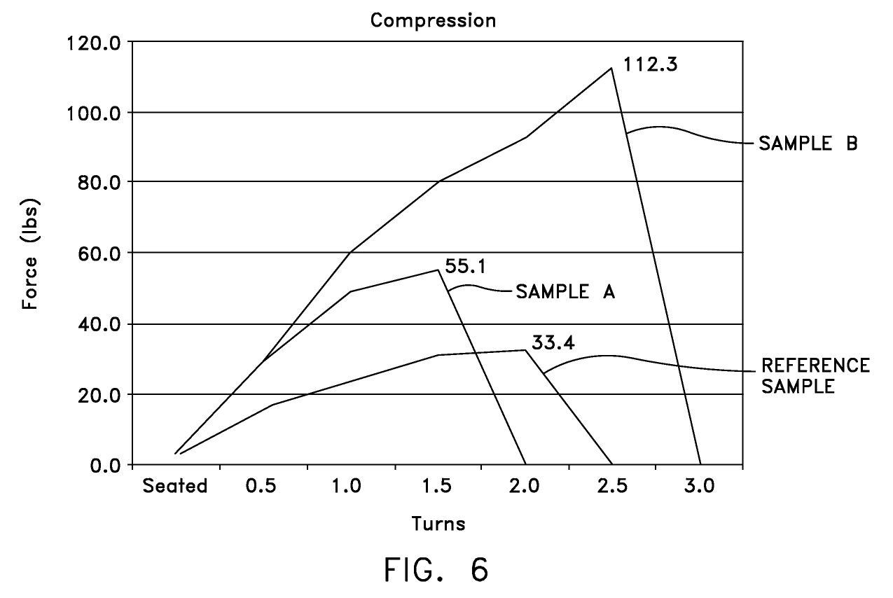

The reference sample tested was a 7.3 mm×110×16 screw available from Synthes (USA) of Paoli, Pa. The sample A tested was a 7.0 mm×110×16 screw according to an embodiment of the invention described herein. The sample B tested was a 7.0 mm×110×32 screw according to another embodiment of the invention. The numbers 16 and 32 refers to the length of the distal thread portion of the screw in millimeters. The test was conducted using 12.5 pcf closed cell foam material per Shaw et al. methods well known and accepted in the art. This compression data plot is shown in FIG. 6.

What is claimed is:

1. A screw implant for insertion in a bone material having first and second regions, the screw implant comprising:
a shaft including a longitudinal axis, a proximal portion, and a distal portion, the proximal and distal portions having proximal and distal threads thereon respectively, said proximal and distal threads each having minor and major diameters, wherein said minor diameter of the proximal thread is substantially equal to the major diameter of the distal thread;
a threadless medial portion disposed on said shaft between said proximal and distal portions;
with said proximal thread being continuous and said proximal and distal threads having substantially the same thread profiles, and substantially the same thread pitch, wherein when said implant is inserted by rotation into the bone material, said proximal and distal portions threadably engage the first and second regions, respectively, providing compression therebetween.

2. The screw implant of claim 1, wherein said distal thread comprises at least two distal thread starts.

3. The screw implant of claim 1, wherein said distal portion having one or more self-drilling flutes provided at its distal tip.

4. The screw implant of claim 1, wherein said proximal thread is arranged in tapered configuration, wherein the proximal thread defines an angle of about six degrees with respect to the longitudinal axis of the shaft.

5. The screw implant of claim 1, wherein said proximal thread is arranged in tapered configuration, wherein the proximal thread defines an angle of at least six degrees with respect to the longitudinal axis of the shaft.

6. The screw implant of claim 1, further comprising a cannulation extending along the longitudinal axis of the screw implant.

7. The screw implant of claim 1, wherein the implant extends from a proximal face to a distal tip and said proximal face is configured and adapted to receive a driving tool.

8. The screw implant of claim 1, wherein the thread pitch is about 0.04 to 0.10 inches.

9. The screw implant of claim 1, wherein the thread pitch is about 0.071 inches.

10. The screw implant of claim 1, wherein the thread profiles of the proximal thread and the distal threads comprise thread leading surfaces having a slope of about 20 to 30 degrees.

11. The screw implant of claim 1, wherein the thread profiles of the proximal thread and the distal threads comprise thread leading surfaces having a slope of about 25 degrees.

12. The screw implant of claim 1, wherein the thread profiles of the proximal thread and the distal threads comprise thread trailing surfaces having a slope of about 2 to 8 degrees.

13. The screw implant of claim 1, wherein the thread profiles of the proximal thread and the distal threads comprise thread trailing surfaces having a slope of about 5 degrees.

14. The screw implant of claim 1, wherein the thread profiles of the proximal thread and the distal threads comprise:
thread leading surfaces having a slope of about 20 to 30 degrees; and
thread trailing surfaces having a slope of about 2 to 8 degrees.

15. The screw implant of claim 1, wherein the thread profiles of the proximal thread and the distal threads comprise a leading trough curvature radius of about 0.02 to 0.04 inches.

16. The screw implant of claim 1, wherein the thread profiles of the proximal thread and the distal threads comprise a leading trough curvature radius of about 0.031 inches.

17. The screw implant of claim 1, wherein the thread profiles of the proximal thread and the distal threads comprise a trailing trough curvature radius of about 0.005 to 0.015 inches.

18. The screw implant of claim 1, wherein the thread profiles of the proximal thread and the distal threads comprise a trailing trough curvature radius of about 0.008 inches.

19. The screw implant of claim 1, wherein the thread profiles of the proximal thread and the distal threads comprise:
a leading trough curvature radius of about 0.02 to 0.04 inches; and
a trailing trough curvature radius of about 0.005 to 0.015 inches.

20. The screw implant of claim 1, wherein the thread profiles of the proximal thread and the distal threads further comprise a thread crest width of about 0.002 to 0.008 inches.

21. The screw implant of claim 14, wherein the thread profiles of the proximal thread and the distal threads further comprise a thread crest width of about 0.002 to 0.008 inches.

22. The screw implant of claim 19, wherein the thread profiles of the proximal thread and the distal threads further comprise a thread crest width of about 0.002 to 0.008 inches.

23. A screw implant for insertion in a bone material having first and second regions, the screw implant comprising:
a shaft including a longitudinal axis, a proximal portion, and a distal portion, the proximal and distal portions having proximal and distal threads thereon respectively, said proximal and distal threads each having minor and major diameters, wherein said minor diameter of the proximal thread is substantially equal to the major diameter of the distal thread;
a threadless medial portion disposed on said shaft between said proximal and distal portions;
with said proximal thread being continuous and said proximal and distal threads having substantially the same thread pitch, substantially the same thread leading surface slope of about 25 degrees, substantially the same thread trailing surface slope of about 5 degrees, substantially the same leading trough curvature radius of about 0.031 inches, substantially the same trailing trough curvature radius of about 0.008 inches, and substantially the same thread crest width of about 0.071 inches, wherein when said implant is inserted by rotation into the bone material, said proximal and distal portions threadably engage the first and second regions, respectively, providing compression therebetween.
